(19) United States
(12) Patent Application Publication
(10) Pub. No.: US 2025/0381229 A1
(43) Pub. Date: Dec. 18, 2025

(54) HYDROGEL COMPOSITIONS CONTAINING CELLULAR PRODUCTS

(71) Applicants: LIVEKIDNEY.BIO LTD, Tel Aviv (IL); ORGENESIS INC., Germantown, MD (US)

(72) Inventors: Masha SIMANOVSKY, Mazkeret Batya (IL); Shimon AMSELEM, Rehovot (IL); Alon YAAR, Ramat Hasharon (IL); Dotan UZI, Kfar-Rut (IL); Svetlana ADAMSKY, Gedera (IL)

(73) Assignees: LIVEKIDNEY.BIO LTD, Tel Aviv (IL); ORGENESIS INC., Germantown, MD (US)

(21) Appl. No.: 18/876,241

(22) PCT Filed: Jun. 20, 2023
(86) PCT No.: PCT/IL2023/050636

§ 371 Date: Dec. 18, 2024

Related U.S. Application Data
(60) Provisional application No. 63/353,637, filed on Jun. 20, 2022.

(51) Int. Cl. A61K 35/28; A61K 9/06; A61K 35/51; A61K 47/36; A61K 47/38; A61P 37/06

(52) U.S. Cl. CPC A61K 35/28; A61K 9/06; A61K 35/51; A61K 47/36; A61K 47/38; A61P 37/06

(57) ABSTRACT

Described herein are hydrogel compositions comprising hyaluronic acid or a pharmaceutically acceptable salt thereof, hydroxypropyl methyl cellulose and a plurality of mesenchymal stromal cells.

Additionally described herein are compositions for treatment, and methods for treatment of a disease, comprising administering to a patient in need thereof a therapeutically effective amount of a composition comprising hyaluronic acid or a pharmaceutically acceptable salt thereof, hydroxypropyl methyl cellulose and a plurality of mesenchymal stromal cells.

### CROSS REFERENCE TO RELATED APPLICATIONS

Benefit is claimed to U.S. Provisional Patent Application No. 63/353,637, filed on Jun. 20, 2022, the contents of which are incorporated by reference herein in their entirety.

### FIELD

Provided herein are compositions comprising cellular products formulated in hydrogels and their uses for treatment of diseases.

### BACKGROUND

Mesenchymal stromal (stem) cells (MSCs) are stem cells that demonstrate robust immunoregulatory properties. MSCs can affect many different immune cell subtypes of both the innate and adaptive immune systems. Multiple laboratory and clinical studies have demonstrated that MSCs can suppress proliferation of T-cells, B-cells, natural killer cells and dendritic cells in a dose-dependent manner. Furthermore, MSCs have the ability to direct macrophages to a more immunotolerant (M2) phenotype that is characterized by alternative activation. Cytokine secretion profiles of T and B-cells are also significantly altered to a less inflammatory and less fibrosis-inducing phenotype by incubation with MSCs, which can further contribute to their observed immunosuppressive properties.

### SUMMARY

Described herein are hydrogel compositions comprising hyaluronic acid or a pharmaceutically acceptable salt thereof, hydroxypropyl methyl cellulose and a plurality of mesenchymal stromal cells.

Additionally described herein are compositions for treatment, and methods for treatment of a disease comprising administering to a patient in need thereof a therapeutically effective amount of a composition comprising hyaluronic acid or a pharmaceutically acceptable salt thereof, hydroxypropyl methyl cellulose and a plurality of mesenchymal stromal cells.

The foregoing and other objects, features, and advantages will become more apparent from the following detailed description, which proceeds with reference to the accompanying figures.

### BRIEF DESCRIPTION OF THE DRAWINGS

FIGS. 1A and 1B depict micrographs showing live/dead assay results of MSC in various formulations of hydrogel versus medium;

FIGS. 1C and 1D depict graphs showing results of an in vitro XTT test showing cell viability in various media at 24 hours (1C) and 48 hours (1D);

FIG. 2A shows a graph of presence of markers indicative of lupus: ANA animals of various treatment groups of a lupus model;

FIGS. 2B-2G show graphs of relative expression as determined by PCR of inflammatory or fibrotic markers in various treatment groups of a lupus model, using an MRL/lpr mouse strain administered different types of subcutaneous hydrogel treatments, with or without cells; and

FIG. 2H shows presence of markers indicative of lupus: double stranded DNA marker in animals of various treatment groups of a lupus model.

### DETAILED DESCRIPTION

### I. Abbreviations

2D 2-Dimensional
3D 3-Dimensional
A260/A280/A320 Absorbance at 260 nm/280 nm/320 nm (Optical Density)
AF Amniotic Fluid
ANA Anti-Nuclear Antibody
Arg-1 Arginase-1
BUN Blood Urea Nitrogen
CaCl2 Calcium Chloride
CCL2 Chemokine (C-C motif) ligand
COL1A Collagen Type 1 Alpha
Cx43 Connexin 43
CXCL1 Chemokine (C—X—C motif) ligand 1
DDW Double Distilled Water
DPBS Dulbecco's Phosphate Buffered Saline, without Ca and Mg
ds-DNA Double Stranded DNA
EVs Extracellular Vesicles
FN1 Fibronectin 1
GFR Glomerular Filtration Rate
H&E Hematoxylin & Eosin
HA Hyaluronic Acid
hFF Human Foreskin Fibroblasts
HG Hydrogel
hMSC Human Mesenchymal Stromal Cell
HPBCD HydroxyPropyl-Beta-CycloDextrin
HPMC Hydroxypropylmethyl Cellulose
IC Intracapsular
IFN Interferon
IL Interleukin
IV Intravenous
min Minutes
MSC Mesenchymal Stromal Cell
N-GAL Neutrophil Gelatinase-Associated Lipocalin
ND Not Done
Os Collagen spark
P Passage
PAS Periodic Acid-Schiff
PBS Phosphate Buffered Saline
PO Per os
RAMEB Random Methylated Beta-Cyclodextrin
RPM Revolutions Per Minute
RT Reverse Transcriptase
RT-qPCR Real Time Quantitative PCR
SBECD Sulfo-Butyl-Ether CycloDextrin
SC Subcutaneous
SLE Systemic Lupus Erythematosus
TGFß Transforming Growth Factor Beta
TNF-α Tumor Necrosis Factor alpha
UC Umbilical Cord
UC-MSC Umbilical Cord-derived Mesenchymal Stromal Cell
UPW Ultra-Pure Water
WFI Water for Injection
XTT (2,3-bis-(2-methoxy-4-nitro-5-sulfophenyl)-2H-tetrazolium-5-carboxanilide) tetrazolium-based compound

### II. Terms

Unless otherwise noted, technical terms are used according to conventional usage. Definitions of common terms in molecular biology can be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

In case of conflict, the present specification, including explanations of terms, will control. In addition, all the materials, methods, and examples are illustrative and not intended to be limiting.

### III. Overview of Several Embodiments

Provided herein are advantageous hydrogel-based compositions, comprising MSC, hyaluronic acid or a salt thereof, preferably sodium salt, and HPMC. These compositions, when administered to animals in disease-states, improved markers of inflammation and reduced markers of fibrosis, especially in a Lupus (lupus) disease state. Such compositions can be used in treating humans, in particular, humans suffering from kidney disease. Methods for treating disease are also provided herein, especially kidney diseases associated with fibrosis and inflammation. Methods for treating lupus nephritis are described herein.

It was found by the inventors that the use of small amounts of HPMC in hyaluronate-based hydrogel compositions improved the characteristics of the hydrogel and the therapeutic potential of the hydrogels. Addition of other excipients to hyaluronate-based hydrogel compositions did not improve, and even decreased the hydrogel characteristics.

In treating disease according to an embodiment, the number of cells, preferably MSCs, per administration of treatment to a human subject in need thereof, is 0.5×10⁶-2×10⁸ cells/kg of subject body weight.

It is suggested that in addition to MSCs, other therapeutic cells may be administered to a patient in need thereof using formulations, comprising hyaluronate and HPMC, to a patient in need thereof. According to an embodiment, the therapeutic cell is an epithelial cell.

In treating a disease according to an embodiment, a composition is administered in an amount of between 0.5 milliliter (ml) and 4.0 ml per administration. Optionally, the composition is administered in an amount of 1.0 ml per administration.

According to an embodiment, therapies described herein are administered subcutaneously to a patient in need thereof. Alternatively, the therapies may be administered locally to the organ being treated. Optionally, the organ being treated is the kidney, and the therapy may be administered to the sub-capsular space of the kidney. The hydrogel therapy administered may be administered once or may be repeated. Optionally, the hydrogel is administered once every year, twice a year, or once every two years. The hydrogels described herein have been shown to provide a paracrine effect over extended periods of time.

According to an embodiment, a method for treatment is described, in which a patient is administered a hydrogel therapy described herein, then is tested for presence of an inflammatory marker, a fibrotic marker or a disease-indicating marker. After such testing a physician may decide to re-administer treatment based on presence or absence of such a marker.

Inflammatory disease, particularly chronic inflammatory diseases, are caused by actions of the immune system, involve inappropriate or excessive activation of certain T-cells, expression of regulatory cytokines and chemokines, loss of immune tolerance, and the like. Examples of autoimmune and/or chronic inflammatory diseases are multiple sclerosis, inflammatory bowel diseases (IBD), joint diseases such as rheumatoid arthritis, and systemic lupus erythematosus. Some of these diseases are rather organ/tissue-specific as follows: intestine (Crohn's Disease), skin (psoriasis), myelinated nerves (multiple sclerosis or MS), pancreatic islet or β cells (insulin dependent diabetes mellitus (IDDM) or Type I Diabetes), salivary glands (Sjogren's disease), skeletal muscle (myasthenia gravis), the thyroid (Hashimoto's thyroiditis; Graves' Disease), the anterior chamber of the eye (uveitis), joint tissue (rheumatoid arthritis), and various cardiovascular diseases.

Progressive scarring (fibrosis) is a pathological feature of many chronic inflammatory diseases and is an important cause of morbidity and mortality worldwide. Fibrosis is characterized by the accumulation of excess extracellular matrix components (e.g., collagen, fibronectin) that forms fibrous connective tissue in and around an inflamed or damaged tissue. Fibrosis may cause overgrowth, hardening, and/or scarring that disrupts the architecture of the underlying organ or tissue. While controlled tissue remodeling and scarring is part of the normal wound healing process promoted by transdifferentiation of fibroblasts into myofibroblasts, excessive and persistent scarring due to severe or repetitive injury or dysregulated wound healing (e.g., persistence of myofibroblasts) can eventually result in permanent scarring, organ dysfunction and failure, and even death.

Fibrotic changes can occur in vascular disorders (e.g., peripheral vascular disease, cardiac disease, cerebral disease) and in all main tissue and organ systems (e.g., lung, liver, kidney, heart, skin). Fibrotic disorders include a wide range of clinical presentations, including multisystemic disorders, such as systemic sclerosis, multifocal fibrosclerosis, and organ-specific disorders, such as pulmonary, liver, and kidney fibrosis Non-limiting examples of fibrotic disorders or fibrotic diseases include pulmonary fibrosis, idiopathic pulmonary fibrosis, cystic fibrosis, liver fibrosis (e.g., cirrhosis), cardiac fibrosis, endomyocardial fibrosis, vascular fibrosis (e.g., atherosclerosis, stenosis, restenosis), atrial fibrosis, mediastinal fibrosis, myelofibrosis, retroperitoneal fibrosis, progressive massive fibrosis (e.g., lungs), chronic kidney disease, nephrogenic systemic fibrosis, Crohn's disease, hypertrophic scarring, keloid, scleroderma, systemic sclerosis (e.g., skin, lungs), athrofibrosis (e.g., knee, shoulder, other joints), Peyronie's disease, Dupuytren's contracture, adhesive capsulitis, organ transplant associated fibrosis, ischemia associated fibrosis, or the like.

Diseases of the kidney may be treated by administering, preferably through the subcutaneous route, a hydrogel composition described herein.

Hydrogels described herein preferably comprise between 0.5% and 5% hyaluronate, by weight, or a pharmaceutically acceptable salt thereof. Preferably, the salt is a sodium salt. Preferably, the amount of hyaluronate by weight in a hydrogel is between 1% and 2% hyaluronate, most preferably 1.5%.

Hydrogels described herein preferably comprise between 0.01% and 0.1% HPMC, by weight, most preferably 0.05% HPMC by weight.

The following examples are provided to illustrate certain particular features and/or embodiments. These examples should not be construed to limit the disclosure to the particular features or embodiments described.

### EXAMPLES

### Example 1

### Preparation of Hydrogel-Based Cellular Formulations, General Description.

Commercial ready-to-use hydrogels were used as is or diluted with the relevant medium as indicated below. For powder-based hydrogels, pharmaceutical grade hydrogels were prepared at room temperature by adding the powder gradually to water or aqueous buffer as indicated below and were gentle mixed with a magnetic stirrer to avoid air bubble formation until all the powder was completely dissolved and the gel was formed.

Hydrogel-based cellular formulations were prepared as follows: Hydrogel was brought to room temperature. Human Mesenchymal Stromal Cells (hMSC) suspension in culture medium was prepared according to number of cells desired. hMSC source: was Promo cells cat #C-12977 [Human Mesenchymal Stem Cells from Adipose Tissue (hMSC-AT)], Umbilical cord hMSC were purchased from OrganaBio. Media used were MSC Growth Medium 2, Promo cells, cat. no. C-28009, lot 472M031; 10% supplement, (proprietorial content), Promo cells, cat. no. C-28009, lot 472M031; 1% Penicillin-Streptomycin Solution, Biological Industries, cat. no. 03-031-1B and CGM, OrganoBio. Hydrogel was added to cell suspension in growth medium and the mixture was gently pipetted up and down 5-10 times to mix thoroughly. The mixture was then transferred to a multi-well plate. The multi-well plate was gently stirred/tilted to ensure an even coating on the bottom of each well.

The multi-well plate was placed in an incubator. Cell viability and proliferation were tested after 24, 48, and 72 hours.

### Example 2

Preparation of Hyaluronic Acid Based Hydrogels, with or without Additives.

Hyaluronic Acid based hydrogels, with cells, were prepared using the general process of Example 1.

In particular, hyaluronate based hydrogel, HY-50 hydrogel, provided by Dechra Pharmaceuticals, (UK), was obtained, having the following ingredients: Sodium hyaluronate (17 mg), sodium chloride (7.57 mg), disodium phosphate heptahydrate (3.78 mg), sodium dihydrogen phosphate monohydrate (0.45 mg), WFI qs to 1 ml.

To prepare hyaluronate based hydrogel with hydroxypropylmethyl cellulose (HPMC) as an additive, HPMC was obtained from Ashland Global Specialty Chemicals (Delaware, USA), as Benecel E5, was diluted to a 0.5% solution by adding 0.1 g of HPMC to 20 ml PBS, and incubating at 37° C. for 10 minutes, and added to the hyaluronate based hydrogel described above, in a ratio of 90% hydrogel to 10% HPMC 0.5% solution.

### Example 3

### Testing of Cell Viability Using XTT

XTT is a test used to determine cell viability and/or proliferation under various conditions. XTT ((2,3-bis-(2-methoxy-4-nitro-5-sulfophenyl)-2H-tetrazolium-5-carboxanilide) tetrazolium-based compound) Cell Viability Kit by Cell Signaling Technology (Massachusetts, USA) was used to test viability of MSC cells in hydrogels prepared according to Example 2, with or without HPMC additive.

Cell seeding was carried out as follows: 350 μL hydrogel and 5×10³ cells suspended in 650 μL medium are mixed in a 1.5 mL eppendorf tube and vortexed for 1 second. 100 μL of the 3D hydrogel mixture was added to each well in 96-well plate. At least three repetitions were performed per sample, and the samples were incubated at 37° C. for the desired time, either 24, 48, or 72 hours.

XTT detection solution was prepared by thawing reagents shortly before testing, then warmed to 37° C. with gentle mixing. Electron coupling solution was added to XTT Reagent (1:50 volume ratio) to prepare the XTT detection solution, then 50 μL XTT detection solution was added to each well of 96-well plate. The plate was returned to the incubator for 3 hours, and absorbance at 450 nm was read.

Cells in medium alone, without hydrogel, were also analyzed to determine their viability in comparable conditions at each time point. The results of the XTT analysis is shown in Table 2 below for cells in hydrogel as prepared in Example 2, with or without HPMC. The letter S indicates that the cell viability was the same in the hydrogel as in the control cells in medium. The letter L indicates that the cell viability was lower in the hydrogel than in the control cells in medium. The letter H indicates that cell viability was higher in hydrogel than in the control cells in medium. ND indicates that no data is available.

TABLE 2

| Hydrogel Type | 24 h | 48 h | 72 h |
|---|---|---|---|
| Hyaluronate based | S | S | L |
| Hyaluronate based | H | H | ND |
| with HPMC | | | |

Surprisingly, hyaluronate based hydrogel with HPMC increased cell viability relative to medium alone.

Other solutions were used to further increase cell viability of hyaluronate based gels, in amount of 10% of the hydrogel, including sodium alginate solution (1% sodium alginate in solution), sulfobutylether-beta cyclodextrin (20% in solution), poloxamer 407 (10% in solution) and Collagen syrup (10% in solution). None of these agents were successful in increasing cell viability beyond that shown in medium alone.

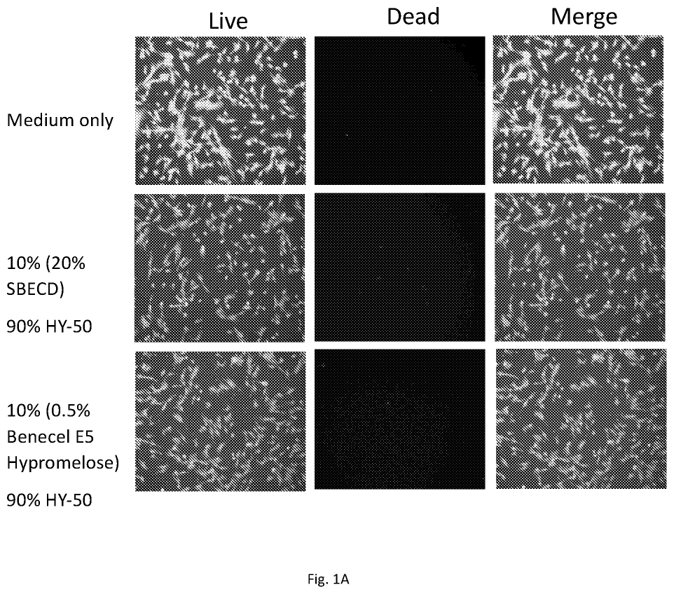

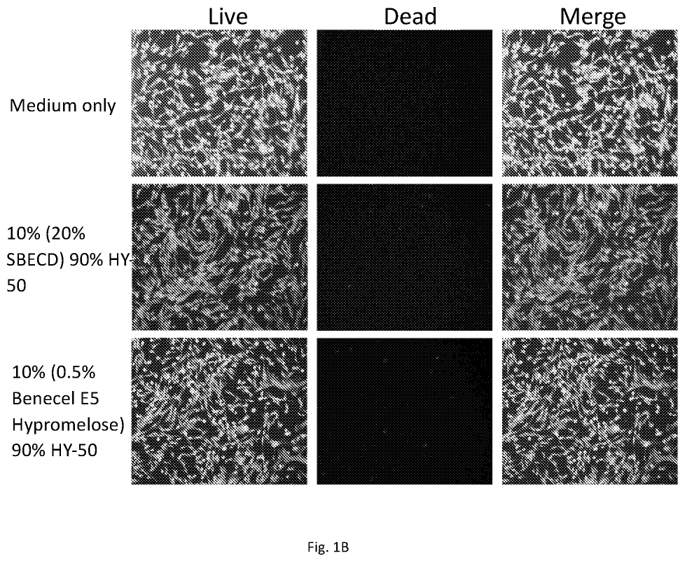

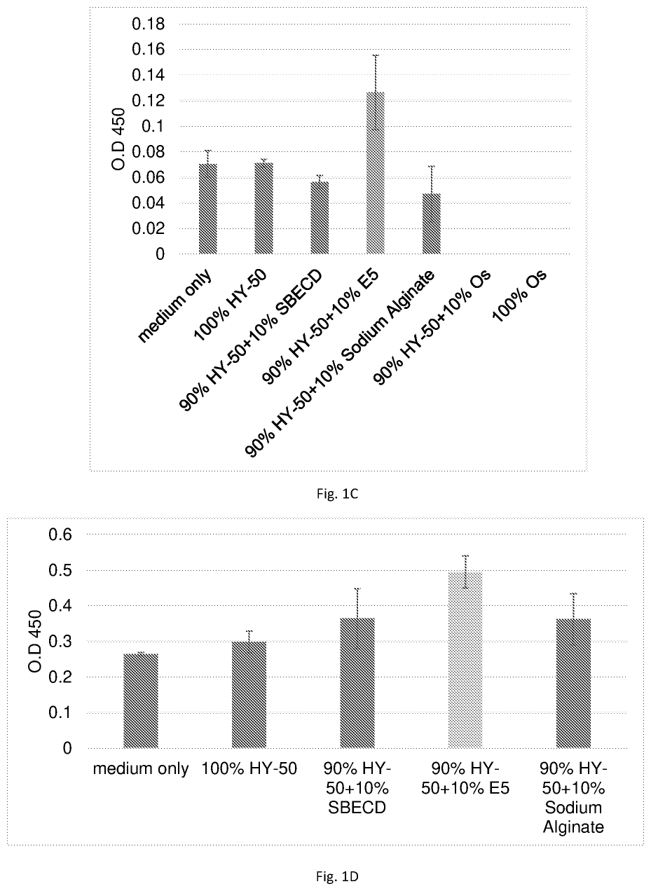

FIG. 1C depicts the XTT results described above after 24 hours, and FIG. 1D depicts the XTT results after 48 hours. In the figures, HY-50 is a reference to the hydrogel, and E5 to HPMC.

### Example 4

### Testing of Cell Viability Using Live/Dead Viability/Cytotoxicity Assay

The Live/dead viability/cytotoxicity assay is based on the simultaneous quantification of live and dead cells with two fluorescent probes (Calcein AM and Ethidium homodimer (EthD-1)) that measure defined parameters of cell viability, namely plasma membrane integrity and intracellular esterase activity.

Method principle: Living cells are characterized by the presence of intracellular esterase activity, determined by the enzymatic conversion of the virtually nonfluorescent cell-permeant Calcein AM to the intensely fluorescent Calcein. The polyanionic dye calcein

is almost completely retained within living cells, producing an intense uniform
green fluorescence in living cells (excitation approximately 495 nm/emission approximately 515 nm). EthD-1 enters cells with compromised membranes and undergoes a 40-fold enhancement of fluorescence upon binding to nucleic acids, thereby producing a bright red fluorescence in dead cells (excitation approximately 495 nm/emission approximately 635 nm). EthD-1 is almost completely excluded by the intact plasma membrane of living cells. The determination of cell viability depends on the physical and biochemical properties of cells. In the model below, 5000 umbilical cord MSCs were seeded in each well in a 96-well plate.

### Cell Seeding in 3D Hydrogel

a. Add 350 μL hydrogel and 1×10⁴ cells suspended in 650 μL medium in a 1.5 mL eppendorf tube.
b. Vortex for 1 sec
c. Add 100 μL of the 3D hydrogel mixture to each well in 96-well plate.
d. Incubate in 37° C. for the desired time.

### Reagent Preparation

a. Thaw reagents just before experiment.
b. Add 5 μL Calcein AM (Component A) and 20 μL ethidium homodimer-1 (Component B) to 10 mL DPBS to create staining solution. *Note: Consider adding less Calcein to epithelial cells
c. Remove medium from cells
d. Add 150 μL of the staining solution directly to cells.
e. Incubate 30 minutes at 20-25° C. Cover or wrap plate in aluminum foil to protect from light during the incubation period.
f. Image cells: Calcein AM excitation/emission-494/517 nm, Ethidium homodimer-1 excitation/emission-528/617 nm.

FIG. 1A depicts results of the live/dead assay after 24 hours and FIG. 1B depicts results of the live/dead assay after 48 hours, comparing cells in medium only, cells in hyaluronic acid hydrogel enriched with 10% solution of SBECD (designated 10% (20% SBECD) 90% HY-50) as described in examples 2 and 3, and in hyaluronic acid hydrogel enriched with 10% solution of 0.5% HPMC (designated 10% (0.5% Benecel E5 Hypromelose) 90% HY-50), as described in example 2.

As can be seen in the figures, HPMC hydrogels performed better than the SBECD based hydrogel.

### Example 5

### In Vivo Study of Hydrogels Comprising MSCs for Treatment of Lupus, in Particular Lupus Nephritis

The MRL/lpr mouse strain was generated by intercrossing four different strains of mice (LG, B6, AKR and C3H). MRL/lpr mice are unique among lupus strains in that they develop a full panel of lupus autoantibodies (ANA, anti-dsDNA, anti-Sm, anti-Ro and anti-La) and have additional lupus manifestations including arthritis, cerebritis, skin rash and vasculitis. The MRL/lpr mouse is used extensively for assessments of candidate treatments for lupus due to the more rapid onset and the multiple manifestations of disease. (Richard & Gilkeson, 2018).

The MRL/lpr mouse strain was used for determining effect of MSC mixed with hydrogel compositions as described in Example 2 in treating lupus and various symptoms and manifestations thereof.

At the beginning of the experiment, on Day −1/0, female MRL/lpr mice and wild type (WT) MRL, age 8 weeks, (weight of about 25-30 g each mouse) underwent transdermal Glomerular Filtration Rate (GFR) measurement, serum and urine collection. At week 14, the MRL/lpr mice were divided into seven groups as listed in Table 3. Group 10 consisted of WT MRL mice and served as a control. IC indicates intracapsular administration, SC indicates subcutaneous administration and PO indicates per os administration. The volume of composition administered in each of the groups was 200 microliters per administration.

TABLE 3

| Group | Number of mice | Treatment |
|---|---|---|
| 2 | 8 | single SC MSC injection 12 × 10⁶ cells |
| | | (hyaluronic acid-HPMC hydrogel) |
| 3 | 8 | single SC MSC injection 12 × 10⁶ cells |
| | | (hyaluronic acid hydrogel) |
| 6 | 5 | Vehicle control SC (hyaluronic acid- |
| | | HPMC hydrogel) |
| 7 | 4 | Vehicle control SC (hyaluronic acid |
| | | hydrogel) |
| 10 | 4 | none |

The following clinical parameters were assessed:
Urine: measure proteinuria by urine stick: twice a week (from week 10)
Weight: twice a week
Lymph nodes visual inspection (score swelling 0, 1, 2, 3)): once a week
Skin rash visual inspection (score rash development 0, 1, 2, 3)): once a week
Arthritic symptoms (general global impression): once a week
Serum: Creatinine, BUN, albumin: day 0, day of sacrifice
GFR: day 0, day of sacrifice

At the completion of the study, the following tests were carried out: Determination of spleen size and weight, scoring and weight of lymph nodes

The following safety parameters were examined: Changes in body weights; food consumption; skin, fur, eyes, mucous membranes, occurrence of secretions and excretions (e.g. diarrhea) and autonomic activity (e.g. lacrimation, salivation, piloerection, unusual respiratory pattern). Changes in gait, posture and response to handling, as well as the presence of bizarre behavior, tremors, convulsions, sleep & coma, morbidity and mortality. Immunomodulatory effects of MSCs in SLE (serum): anti-dsDNA and antinuclear antibodies (ANA) by ELISA. The autoantibodies against ds-DNA in murine serum, were analyzed using a CUSABIO ELISA assay, (Mouse Anti-dsDNA IgG Antibody ELISA Kit) utilizing serum from tested animals according to the manufacturer's instructions.

The anti-nuclear Antibody (IgG) levels in murine serum, were analyzed using a CUSABIO ELISA assay, (Mouse anti-nuclear antibody (IgG) ELISA Kit) utilizing serum from tested animals according to the manufacturer's instructions.

No significant impact of any of the treatments on GFR, body weight, or spleen weight was shown. A reduction in proteinuria score was shown in the subcutaneous hyaluronic acid hydrogel group.

RNA samples were prepared as follows for determination of cytokine level via PCR. Samples (half of whole kidney) were stored at −80° C. The kidney tissue was placed in tube containing one metal bead and 700 μL TRI Reagent, and ground for 5 min at high speed in the grinding machine. The tube was then centrifuged at 300×g for 5 min. 500 μL of upper fluid was transfer to a new tube and 500 μL of ethanol was added. Total RNA was isolated from samples by DirectZol miniprep kit (Cat no. R2072) according to manufacturer's protocol. The elution volume was 50 μL. Samples were stored at −80° C.

The RNA volume required for synthesis of 500 ng cDNA samples was calculated according to the RNA concentration data obtained from the Nanodrop. cDNA Synthesis was performed by using “High-Capacity cDNA Reverse Transcription” kit from Applied Biosystems Cat #4368814.

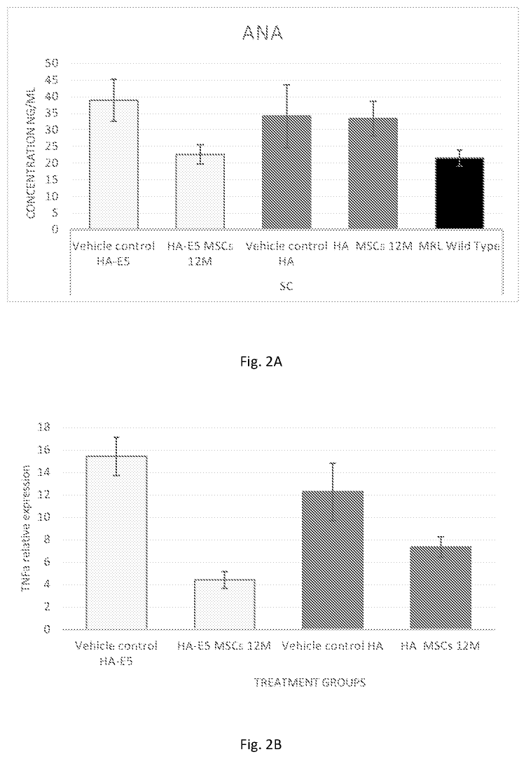

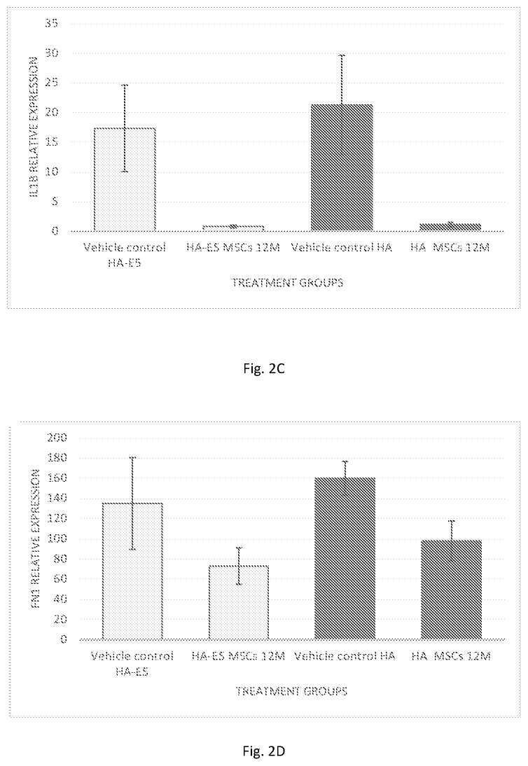

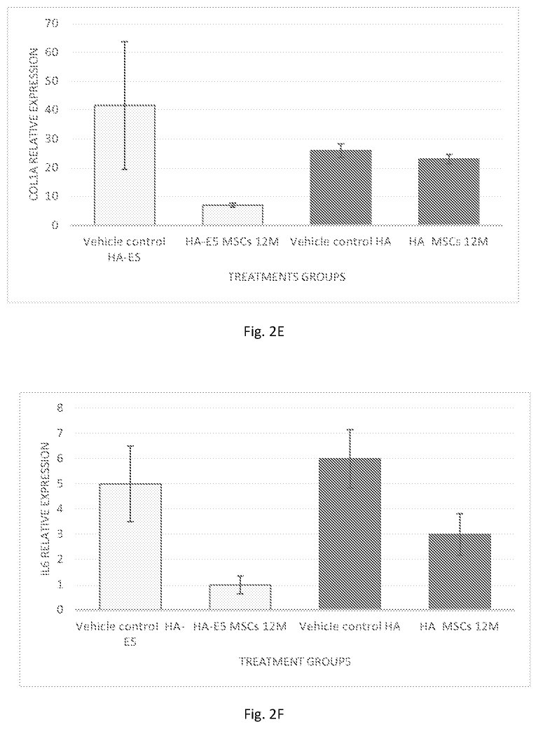

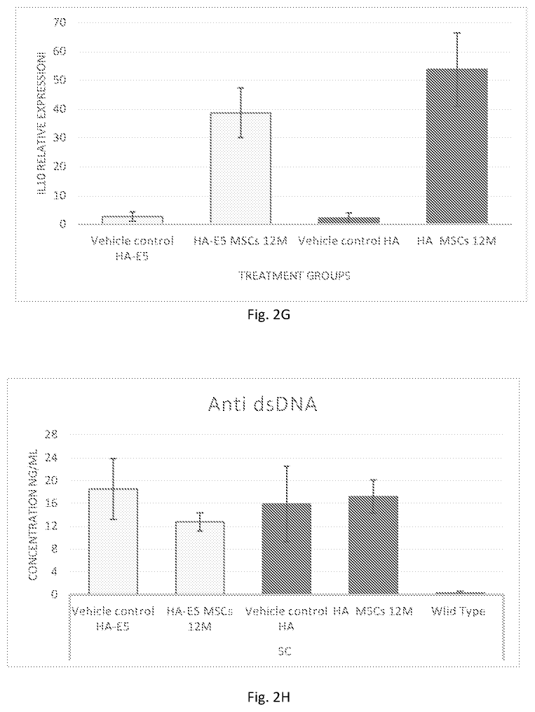

Relative gene expression for various cytokines/chemokines were determined by PCR and are shown in graphs in FIGS. 2B-2G. In the graphs, “Vehicle control HA-E5” indicates that the hyaluronic acid, HPMC composition, described above without cells was used. “HA-E5 MSCs 12M” indicates that 12 million cells with the hyaluronic acid, HPMC composition was used. “Vehicle Control HA” indicates that the hyaluronic acid composition (without HPMC) was used, without cells. “HA MSCs 12M” indicates that the hyaluronic acid composition (without HPMC) was used with 12 million cells. Marker levels indicative of lupus, ANA and ds-DNA are shown in FIGS. 2A and 2H respectively.

In summary, MSC injected subcutaneously in hydrogel formulations increased local relative expression of the anti-inflammatory cytokine IL-10, and reduced the expression of pro-inflammatory cytokines TNFα, IL-1β and IL-6 and of the fibrosis markers, FN1 and COL1A. It is suggested that MSC injected SC in hydrogel compositions as described herein, comprising HPMC, can induce an anti-inflammatory response via a paracrine effect. In addition, as shown in FIGS. 2A and 2H, MSC injected SC in hydrogel compositions as described herein, comprising HPMC, can reduce presence of lupus-related markers, indicating potential therapeutic value of these compositions.

Described herein according to an embodiment, is a hydrogel composition comprising hyaluronic acid or a pharmaceutically acceptable salt thereof, hydroxypropyl methyl cellulose and a plurality of mesenchymal stromal cells (MSCs). Optionally, the MSCs are of umbilical cord or adipose origin. Optionally, salt of hyaluronic acid is sodium hyaluronate. Optionally, the hydrogel comprises between 0.5-5 percent, by weight of hyaluronic acid or a pharmaceutically acceptable salt thereof. Optionally, the hydrogel comprises 1.5 percent, by weight of hyaluronic acid or a pharmaceutically acceptable salt thereof. Optionally, the hydrogel comprises between 0.01 and 0.1 percent, by weight of HPMC. Optionally, the hydrogel comprises 0.05 percent, by weight of HPMC. Optionally, the composition comprises up to 6.0×10⁷ cells per ml. Optionally, the composition is for use in treatment of an inflammatory or fibrotic disease of a kidney in a patient.

Additionally described herein is a method for treatment of an inflammatory or fibrotic disease of a kidney in a patient comprising administering to a patient in need thereof a therapeutically effective amount of hydrogel composition comprising hyaluronic acid or a pharmaceutically acceptable salt thereof, hydroxypropyl methyl cellulose and a plurality of mesenchymal stromal cells (MSCs). Optionally, the disease is an inflammatory disease. Optionally, the disease is a fibrotic disease. Optionally, the composition is administered through the subcutaneous or intradermal route. Optionally, the composition is administered through the subcapsular route. Optionally, the therapeutically effective amount is between 0.5×10⁶ and 2×10⁸ cells per kilogram of body weight of the patient. Optionally, the composition is administered in a volume of between 1.5-4 ml per administration. Optionally, the composition is administered at intervals of between six months and two years. Optionally, the inflammatory disease is selected from the group consisting of: CKD and lupus nephritis. Optionally, the fibrotic disease is selected from the group consisting of: kidney fibrosis and chronic kidney disease, and nephrogenic systemic fibrosis. Optionally, the disease is lupus nephritis.

Additionally described herein is a method for treating a disease, selected from the group comprising: osteoarthritis, urinary incontinence, atopic dermatitis, psoriasis, or any combination thereof, the method comprising administering to a patient in need thereof a therapeutically effective amount of hydrogel composition comprising hyaluronic acid or a pharmaceutically acceptable salt thereof, hydroxypropyl methyl cellulose and a plurality of mesenchymal stromal cells (MSCs).

In one embodiment, the disease is osteoarthritis. In another embodiment, the disease is urinary incontinence. In another embodiment, the disease is atopic dermatitis. In another embodiment, the disease is psoriasis.

Additionally described herein is a method for treating aging comprising administering to a patient in need thereof a therapeutically effective amount of hydrogel composition comprising hyaluronic acid or a pharmaceutically acceptable salt thereof, hydroxypropyl methyl cellulose and a plurality of mesenchymal stromal cells (MSCs). A skilled artisan would understand the term “aging” as the time-related deterioration of the physiological functions necessary for survival and fertility. In humans, aging represents the accumulation of changes in a human being over time and can encompass physical, psychological, and social changes. In some embodiments, aging comprises wrinkles, dullness of skin, uneven skin tone, dry skin, blotchiness and age spots, rough skin texture, visible pores or any combination thereof.

Additionally described herein is a method for treating a wound comprising administering to a patient in need thereof a therapeutically effective amount of hydrogel composition comprising hyaluronic acid or a pharmaceutically acceptable salt thereof, hydroxypropyl methyl cellulose and a plurality of mesenchymal stromal cells (MSCs). In some embodiments, the wound comprises abrasions (scraping of the outer skin layer), lacerations (a tear-like wound), contusions (swollen bruises due to accumulation of blood and dead cells under skin), concussions (damage to the underlying organs and tissue on head with no significant external wound), stab wounds (trauma from sharp objects, such as knives), skin cuts, surgical wounds (intentional cuts in the skin to perform surgical procedures), gunshot wounds (wounds resulting from firearms), thermal wounds, chemical wounds, bites and stings, electrical wounds, ulcers or any combination thereof.

In one embodiment, the hydrogel composition is administered through the topical route. In another embodiment, the hydrogel composition is administered through the systemic route.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1. A hydrogel composition comprising hyaluronic acid or a pharmaceutically acceptable salt thereof, hydroxypropyl methyl cellulose and a plurality of mesenchymal stromal cells (MSCs).

2. The hydrogel composition according to claim 1 wherein the MSCs are of umbilical cord or adipose origin.

3. The composition according to claim 1 wherein the salt of hyaluronic acid is sodium hyaluronate.

4. The composition according to claim 1, wherein the hydrogel comprises between 0.5-5 percent, by weight of hyaluronic acid or a pharmaceutically acceptable salt thereof.

5. The composition according to claim 4 wherein the hydrogel comprises 1.5 percent, by weight of hyaluronic acid or a pharmaceutically acceptable salt thereof.

6. The composition according to claim 1, wherein the hydrogel comprises between 0.01 and 0.1 percent, by weight of HPMC.

7. The composition according to claim 6 wherein the hydrogel comprises 0.05 percent, by weight of HPMC.

8. The composition according to claim 1, comprising up to 6.0×10⁷ cells per ml.

9. The composition according to claim 1, for use in treatment of an inflammatory or fibrotic disease of a kidney in a patient.

10. A method for treatment of an inflammatory or fibrotic disease of a kidney in a patient comprising administering to a patient in need thereof a therapeutically effective amount of a composition according to claim 1.

11. The method according to claim 10 wherein the disease is an inflammatory disease.

12. The method according to claim 10 wherein the disease is a fibrotic disease.

13. The method according to claim 10 wherein the composition is administered through the subcutaneous or intradermal route.

14. The method according to claim 10 wherein the composition is administered through the subcapsular route.

15. The method according to claim 10 wherein the therapeutically effective amount is between 0.5×10⁶ and 2×10⁸ cells per kilogram of body weight of the patient.

16. The method according to claim 10 wherein the composition is administered in a volume of between 1.5-4 ml per administration.

17. The method according to claim 10 wherein the composition is administered at intervals of between six months and two years.

18. The method according to claim 11 wherein the inflammatory disease is selected from the group consisting of: CKD and lupus nephritis.

19. The method according to claim 12 wherein the fibrotic disease is selected from the group consisting of: kidney fibrosis, and chronic kidney disease, and nephrogenic systemic fibrosis.

20. The method according to claim 18 wherein the disease is lupus nephritis.
